(19) United States
(12) Patent Application Publication
(10) Pub. No.: US 2014/0256661 A1
(43) Pub. Date: Sep. 11, 2014

(54) PHARMACEUTICAL COMPOSITIONS FOR RECTAL ADMINISTRATION

(71) Applicant: David Nigel Armstrong, Lawrenceville, GA (US)

(72) Inventor: David Nigel Armstrong, Lawrenceville, GA (US)

(21) Appl. No.: 14/131,715

(22) PCT Filed: Jul. 12, 2012
(86) PCT No.: PCT/US12/46395

§ 371 Date: May 28, 2014

Related U.S. Application Data
(60) Provisional application No. 61/508,120, filed on Jul. 15, 2011.

(51) Int. Cl. A61K 9/00; A61K 31/496; A61K 31/437; A61K 31/424; A61K 31/7048; A61K 31/65; A61K 31/4164; A61K 31/43

(52) U.S. Cl. CPC A61K 9/0031; A61K 31/4164; A61K 31/496; A61K 31/43; A61K 31/424; A61K 31/7048; A61K 31/65; A61K 31/437
USPC 514/29; 514/398; 514/253.08; 514/152

(57) ABSTRACT

The present invention relates to a pharmaceutical composition, in particular a composition formulated for enema administration, wherein the composition comprises metronidazole or a pharmacologically acceptable derivative thereof in an amount to effectively treat both acute and chronic pouchitis and/or proctitis.

### CROSS-REFERENCE TO RELATED APPLICATION

This application claims priority to U.S. Provisional Patent Application No. 61/508,120, filed on Jul. 15, 2011, the contents of which are hereby incorporated by reference herein for all purposes.

### BACKGROUND OF THE INVENTION

1. Technical Field

The present invention relates to a pharmaceutical composition, in particular a composition formulated for enema delivery, wherein the composition comprises metronidazole or a pharmacologically acceptable derivative thereof in an amount to effectively treat both acute and chronic pouchitis and/or proctitis.

2. Related Art

Inflammatory bowel disease comprises two conditions, ulcerative colitis and Crohn's disease. Both ulcerative colitis and Crohn's disease are chronic inflammatory diseases of the digestive tract, the former restricted to the large intestine and the latter affecting any part of the bowel from mouth to anus. Idiopathic proctitis is often recognized as a separate entity. It usually involves the distal rectum, is most common in young males and is usually self limiting. Etiology is unknown. Proctitis may also arise secondary to radiation, HIV or sexually transmitted disease (Chlamydia, gonococcus syphilis etc).

The principal symptoms of ulcerative colitis are diarrhea and rectal bleeding. Medical treatment of ulcerative colitis is usually treated with corticosteroids (intravenous, orally or topically) with their attendant side-effects. Sulphasalazine and its derivatives (5-aminosalicylic acid) can be used in active disease and are effective in reducing the incidence of relapse, but occasionally with troublesome side effects. Immunosuppressive agents such as azathioprine and 6-mercaptopurine are used in patients not responding to steroids or sulphasalazine and again have adverse effects.

Abdominal colectomy with mucosal protectomy and ileal pouch-anal anastomosis is the preferred treatment for most patients with ulcerative colitis who require surgery. However, a long term complication of such a procedure is pouchitis. Pouchitis is defined as a clinical syndrome of watery, and at times, bloody stool that can be accompanied by urgency, incontinence, abdominal and cramps. Chronic pouchitis is distinguished from acute pouchitis by duration of symptoms for more than 4 weeks. The etiology of pouchitis is unknown but it appears that both a history of ulcerative colitis (rather than Familial Polyposis), and altered bacterial concentrations (relative to the normal ileum) are factors. Currently, there is no satisfactory treatment for patients with chronic pouchitis who fail to respond to antibiotic therapy.

Metronidazole (or “Flagyl”) is a synthetic antibacterial and antiprotozoan antibiotic having the formula 2-methyl-5-nitroimidazole-1-ethanol. The antibiotic has been used for many years in its oral or intravenous form, to treat inflammatory conditions of the colon, rectum, anal canal and perianal region. Oral metronidazole has been traditionally used to treat inflammatory bowel disease including ulcerative colitis, idiopathic proctocolitis, or radiation proctitis. In addition, the oral form is used to treat inflammatory conditions of the perianal region or anal canal such as anal fissures, fistulas, abscess, ulcers or post-surgical wounds. Unfortunately, the use of oral administration of metronidazole for the treatment of pouchitis has been associated with a number of negative side effects, such as, nausea, vomiting, a metallic taste in the mouth, or inflammation of the oral cavity. Additionally, some negative neurological side effects may occur which usually manifest as numbness or tingling of the extremities.

Topical metronidazole has previously been used for a number of skin conditions (e.g. rosacea) or as a topical vaginal preparation in the treatment of vaginal infections (e.g. trichomonas). These preparations are contained in a medium containing alcohol, which would result in stinging and burning when used in the perianal region or in the anal canal. Notably, there is no disclosure of the direct application of metronidazole into the rectum or distal colon as an enema in the treatment of pouchitis.

Thus, there is a clear need for an alternative administering route of effective active agents that provides a safe and effective treatment of pouchitis.

### SUMMARY OF THE INVENTION

The present invention provides a therapeutic method for treating pouchitis comprising locally administering to the ileal pouch, rectum or lower section of the descending colon of a patient in need of such treatment an effective amount of antibiotic compound to reduce the symptoms of pouchitis or proctitis due to ulcerative colitis, idiopathic proctitis, Crohn's proctitis or proctitis secondary to radiation, HIV or other factor. The antibiotic compound may include, but is not limited to metronidazole, ciprofloxacin, amosicillin/clavulanic acid/erythromycin, tetracycline, ritazimin/ciprofloxacin or metronidazole/ciprofloxacin. Preferably, the antibiotic compound is metronidazole or a pharmaceutically acceptable salt thereof.

It is an object of the present invention to locally administer metronidazole to treat certain conditions of the intestinal tract avoiding unwanted side effects caused by oral or intravenous administration. In light of the fact that metronidazole possesses not only anti-bacterial properties, but also anti-inflammatory properties, it may be used for its anti-inflammatory properties in the treatment of pouchitis by a mode of administration that has not been previously explored.

Thus, in one aspect the present invention provides a method of reducing the symptoms of acute or chronic pouchitis or proctitis, the method comprising delivering to the ileal pouch, rectum and/or descending colon of the subject, by the rectal route, a pharmaceutically effective amount of metronidazole or salt thereof to effect an improvement in the symptoms of pouchitis or proctitis including a reduction in the number of daily bowel movements and an improvement in the consistency of the feces, as well as reduced losses of blood and mucus.

In yet another aspect, the present invention provides for a composition formulated for enema delivery and comprising metronidazole or a pharmaceutically acceptable salt thereof in an amount ranging from about 0.1% to 1.5% w/w to ameliorate the symptoms of pouchitis or proctitis.

The enema formulation of the present invention preferably comprises Metronidazole in an amount from about 0.4% to about 0.9% in an aqueous solution in combination with at least one additional component comprising a preservative, viscosity enhancer, co-solvent or buffer.

In a further aspect, the present invention provides for a method for the treatment of ulcerative colitis Crohns disease idiopathic or secondary proctitis comprising the rectal application of a pharmaceutical composition formulated for enema delivery containing metronidazole or pharmaceutically acceptable salt thereof as an active ingredient in combination with suitable excipients and/or diluents, said pharmaceutical composition containing between 0.1% to 1.5% w/w of active ingredient per unitary dose. The concentrations are based on the total weight of the composition.

Preferably the metronidazole is administered to the colon by rectal administration of an enema formulation or rectal foam. The rectal enema formulation is preferably a viscous solution, which may also include preservatives, chelating agents, pH regulators, thickeners, solubilizers, buffers, emulsifiers and/or solvents.

The composition may consist essentially of metronidazole as the active agent. However, a therapeutic amount of at least one other active agent may be added to the composition to add to its effectiveness. Additional active agents that may be added include steroids, e.g. hydrocortisone or a pharmacologically acceptable derivative thereof, analgesic agents, preferably from the amide or ester class such as pramoxine or benzocaine, antimicrobial agents (antibacterial or antiviral), e.g. ciprofloxacin, amoxicillin-clavulonic acid, erythromycin, tetracycline, clindamycin or doxycyclin, substances that either promote skin integrity or inhibits skin breakdown, e.g. vitamin E, aloe, zinc oxide or other barrier cream, anti-inflammatory agents, e.g. a non-steroidal anti-inflammatory agent selected from aminosalicylic acid, ibuprofen, sulindac, piroxicam or diflunisal and antidiarrheal compounds such as a bismuth salt. The additional or supplemental antibiotic or antiviral medications may add to the anti-bacterial spectrum of activity (gram positive, gram negative aerobic or anaerobic, antiviral) of metronidazole.

In yet another aspect, the present invention relates to kits for the treatment of irritable bowel syndrome, proctitis Crohn's disease or pouchitis wherein the kit includes packaging that contains a composition formulated for enema delivery and comprising at least an effective amount of metronidazole in a pharmaceutically acceptable carrier.

In a still further aspect, the present invention provides for the use of metronidazole in the amount from about 0.1% w/w to about 1.5% w/w in the manufacture of a medicament for the treatment of the symptoms of pouchitis or proctitis.

These and other advantages and features of the present invention will be described more fully in a detailed description of the preferred embodiments which follows.

### DETAILED DESCRIPTION OF THE INVENTION

Throughout the instant specification and claims, the following definitions and general statements are applicable.

As used herein, whether in a transitional phrase or in the body of a claim, the terms “comprise(s)” and “comprising” are to be interpreted as having an open-ended meaning That is, the terms are to be interpreted synonymously with the phrases “having at least” or “including at least.” When used in the context of a process, the term “comprising” means that the process includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the desired properties of the compositions described herein, i.e., the process or composition is limited to the specified steps or materials and those which do not materially affect the basic and novel characteristics of the invention.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, “treating” means reducing, hindering or inhibiting the development of, controlling, alleviating and/or reversing the symptoms in the individual to which a combination or composition of the invention has been administered, as compared to the symptoms of an individual not being treated according to the invention. A practitioner will appreciate that the combinations, compositions, dosage forms and methods described herein are to be used in concomitance with continuous clinical evaluations by a skilled practitioner to determine subsequent therapy.

Without wishing to be bound by any particular theory, it is believed that the use of metronidazole by direct application to the diseased or otherwise affected is primarily a local effect. Minimal systemic absorption is observed and therefore systemic side effects are effectively reduced or eliminated. As such, the dose of metronidazole can be altered for specific tissue and applied directly to the diseased or otherwise effected area thereby increasing the efficacy of the medication.

The rectal enemas of the present invention are generally liquid compositions, solutions, emulsions or aqueous suspensions having at least one active ingredient and at least one additional component including preservatives, chelating agents, surfactants, thickeners, thickeners-solubilizers, buffers, co-solvents, or lubricants.

According to one embodiment of the present invention, the pharmaceutical composition takes the form of an enema formulation such as a liquid or foam enema which is rectally administered to the ileal pouch or lower colon/rectum. The enema formulations comprise metronidazole dissolved or dispersed in a suitable flowable carrier vehicle. The formulation can be thickened with one or more thickeners, can contain a buffer, and can also comprise an effective amount of a lubricant such as a natural or synthetic fat or oil, e.g. a tris-fatty acid glycerate or lecithin. Non-toxic non-ionic surfactants can also be included as wetting agents and dispersants. Unit doses of enema formulations can be administered from an enemator, pre-filled bags or syringes. In the case of a pressurized enema formulation the carrier vehicle may also comprise an effective amount of a foaming agent such as n-butane, propane or i-butane. Preferably, the composition does not include an alcohol.

A dosage form of metronidazole adapted for rectal delivery may be complexed with a suspending or thickening agent to increase viscosity and prolong release of the dosage form of metronidazole. Such agents include acrylic acid polymers, preferably carbomers (carboxypolymethylene) which are synthetic high molecular weight acrylic acid polymers crosslinked with polyfunctional moieties such as polyallylsucrose.

Generally, carbomers comprise 50 to 70% carboxylic acid groups. Carbomers are mucoadhesive and adhere to colonic mucus thereby potentially maximizing the metronidazole/carbomer effect on the colonic mucosa. As carbomers adhere strongly to mucus membranes in gel form, they serve as excellent local delivery vehicles for bioactive compounds. Importantly the use of a mucoadhesive additive provides for dispersion in the large intestine and coats the intestinal wall while having the advantage of holding the metronidazole in contact with the inflamed intestinal wall. The invention is therefore a major advance over the oral administration.

In one embodiment of the present invention, the carbomer is Carbopol. Such polymers are commercially available from B.F. Goodrich under the designation Carbopol 420, 430, 475, 488, 493, 910, 934, 934P and the like. In a particularly preferred embodiment the carbomer is Carbopol® 974P. Carbomers are available as fine white powders which disperse in water to form acidic colloidal suspensions of low viscosity. The viscosity of the enema is preferably 5,000 to 70,000 mPas more preferably 10,000 to 40,000 mPas.

Neutralization of these suspensions using a base, for example sodium, potassium or ammonium hydroxides, low molecular weight amines and alkanolamines, results in the formation of a gel like material. The pH is preferably 3.5 to 7.5, especially 6.5 to 7.5.

A suitable % w/w of metronidazole in an enema formulation (based on 100 ml enema solution) is from about 0.1% to about 1.5 w/w, more preferably from about 0.4% to about 0.9% w/w, and most preferably from about 0.5% to about 0.6% w/w.

The following is a description of the present invention by way of example only and is not intended to limit the scope of the invention as defined in the claims.

### Example 1

### Enema Suspension

A suspension composition suitable as an enema formulation containing about 6 g metronidazole by weight was prepared by the following procedure.
1. 200 g of polyethyleneglycol and about 500 g of purified water were combined and placed in a beaker, and mixed with 1.5 g or methyl-4-hydroxybenzoate and 0.2 g or propyl-4-hydroxybenzoate under stirring conditions and at a temperature of about 50° C.; 6 g of metronidazole was added and the temperature adjusted to about 20° C.;
2. 6.0 g of carbomer 974 is mixed with about 100 g of purified water and dispersed under stirring for about 1 minute to form a homogenized mixture; and
3. 1.0 g of sodium hydroxide is dissolved in about 138.2 g of purified water and dispersed under stirring to form a homogenized mixture with the subsequent addition of 2.2 g of potassium hydrogen phosphate; and all three solutions are combined at about room temperature.

### Example 2

### Enema

To prepare an enema formulation, the following ingredients are dissolved or suspended in purified water:

| | | |
|---|---|---|
| Metronidazole | 0.6% | Active |
| Methyl-4-hydroxybenzoate | 0.15% | Preservative |
| Propyl-4-hydroxybenzoate | 0.02% | Preservative |
| Polyethylene glycol | 20.0% | Co-solvent |
| Carbomer | 0.6% | Viscosity enhancer/ |
| | | mucoadhesive |
| Sodium hydroxide | 0.1% | Buffer |
| Potassium hydrogen phosphate | 0.22% | Buffer |

The mixture is neutralized with sodium hydroxide solution (pH, about 7.3) resulting in a clear solution which is made up with water to 100 ml and filled into bottles or other type vials and sealed.

### Example 3

The present invention provides for a composition comprising at least one active agent and a solvent formulated for enema delivery. The composition may include additional components useful for enhancing delivery and adhering to mucosal tissue of the intestinal wall. The following list of components provides alternative choices for active and inactive components:

Active Agent
Range: from about 0.1% w/w to about 1.5% w/w
Active agents may include an anti-biotic or anti-fungal or combination selected from a group consisting of metronidazole, ciprofloxacin, amosicillin/clavulanic acid/erythromycin, tetracycline, ritazimin/ciprofloxacin and metronidazole/ciprofloxacin;

Preservative
Range: from about 0.1% w/w to about 0.3% w/w
Any acceptable preservative could be used such as parabens, benzalkonium chloride, alkyl hydroxyl benzoates, benzoic acid and corresponding salts, methylparaben, benzophenone-4, methylchloroisothiazolinone, and sodium benzoatemethylisothiazolinone;

Viscosity Enhancer
Range: from about 0.5% w/w to about 5% w/w
Polymers (anionic, cationic, non-ionic) and corresponding salts, propylene glycol, soft paraffin, aluminum stearate, polyethylene glycols, hydrogenated lanolin, beeswax, celluloses (alkyl, carboxyalkyl, hydroxyalkyl) and corresponding salts, and gums, such as xanthan, carrageenan, gelatin, karaya, pectin and locust beans gum;

Mucoadhesive
Range: from about 0.5% w/w to about 5.0% w/w
Polymers (anionic, cationic, non-ionic) and corresponding salts, celluloses (alkyl, carboxyalkyl, hydroxyalkyl) and corresponding salts, alginate, and carbomers (carboxypolymethylene);

Buffer
Range: from 0.5% w/w to 2.0% w/w or a sufficient amount to adjust pH to 6.5 to 7.5
Any acceptable buffer system could be used such as sodium chloride, sodium hydroxide, potassium hydrogen phosphate, sodium hydrogen phosphate, potassium hydroxide, potassium chloride, citric acid, sodium acetate, and sodium EDTA;

Surfactant
Range: from about 0.5% to about 10%
Preferred surfactants, including both the foaming and non-foaming type, include sodium laureth sulfate, sodium laureth-13 carboxylate, disodium laureth sulfosuccinate, disodium cocoamphodiacetate, glycol stearate, PEG-150 distearate and mixtures thereof; and

Co-Solvents
Range: up to 50% w/w
Any acceptable co-solvent could be used such as polyethylene glycols, propylene glycols, in addition to the purified water used a the primary solvent. Alcohols are not used because of the negative side effects such as burning and possible systemic effects with other drugs in a subject's system.

Acceptable dosing can occur once a day, every other day, three times a week, or twice a week. It can also occur in divided doses, twice, three, or four times a day. One acceptable dosing schedule is once a day. Initial treatment can continue for up to 2 weeks for an acute condition, or about 4 weeks to about 16 weeks for a chronic condition, or alternatively about 8 weeks to about 12 weeks for a chronic condition. Additionally, patients can receive treatment with a higher dose of the composition until a desired reduced disease state is achieved, and then continue on a lower dose of the composition.

### Example 4

Preliminary clinical trials are conducted with metronidazole in the form of enema using 40 patients of both sexes suffering from pouchitis. Half of the patients are suffering from acute pouchitis and the other half suffering from chronic pouchitis. Patients are divided into four groups and treated with an enema solution product, half with the active agent metronidazole in a dosage amount of about 0.6% in 100 ml of solution and the other half without the active agent (control group). The test is conducted for 30 days.

The test results are determined by using the Pouchitis Disease Activity Index (PDAI) (Sandborn, et. al., Mayo Clinic Proc. 1994, B. 69, pp. 409-415) that provides a standardized definition wherein a score greater than or equal to 7 indicates pouchitis. The obtained results can be further defined by using the Heidelberg Pouchitis Activity Score (PAS) (Heidelberg, et al., Dis Colon Rectum, 2001, V. 44, pp. 487-499) to determine if the symptoms of pouchitis are reduced and provide evidence of the effectiveness of metronidazole.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

That which is claimed is:

1. A composition to ameliorate the symptoms of pouchitis or proctitis and formulated for enema delivery, the composition comprising metronidazole or a pharmaceutically acceptable salt thereof in an amount ranging from about 0.1% to 1.5% w/w.

2. The composition of claim 1, wherein the metronidazole in an amount from about 0.4% to about 0.9% in an aqueous solution.

3. The composition of claim 2, wherein the metronidazole is in combination with at least one additional component comprising a preservative, viscosity enhancer, co-solvent or buffer.

4. The composition of claim 3, wherein the preservative is in an amount from about 0.1% w/w to about 1.5% w/w, the viscosity enhancer is in an amount from about 0.5% w/w to about 5% w/w, and the buffer is in an amount sufficient to adjust pH to between 6.5 and 7.5.

5. The composition of claim 1, wherein metronidazole is the sole active agent in the composition used to ameliorate the symptoms of pouchitis or proctitis.

6. The composition of claim 1, wherein said enema formulation consists essentially of metronidazole at a concentration of about 0.5% to 0.6% w/w in a pharmaceutically acceptable carrier.

7. A therapeutic method of treating pouchitis or proctitis comprising locally administering to the rectum, colon and/or ileal pouch of a patient in need of such treatment a composition formulated for enema delivery comprising an effective amount of an antibiotic compound to reduce the symptoms of pouchitis or proctitis.

8. The method of claim 7, wherein the antibiotic compound is selected from the group consisting of metronidazole, ciprofloxacin, amosicillin/clavulanic acid/erythromycin, tetracycline, ritazimin/ciprofloxacin and metronidazole/ciprofloxacin.

9. The method of claim 7, wherein the antibiotic compound is metronidazole or a pharmaceutically acceptable salt thereof.

10. The method of claim 9, wherein in the metronidazole or a pharmaceutically acceptable salt thereof in an amount ranging from about 0.1% w/w to 1.5% w/w.

11. The method of claim 7, wherein the composition formulated for enema delivery further comprises pharmaceutically acceptable excipients and/or diluents.

12. The method of claim 7, wherein the composition formulated for enema delivery further comprises additional components selected from the group consisting of preservative, viscosity enhancer, mucoadhesive, buffer, surfactant and solvent.

13. The method of claim 7, wherein the composition formulated for enema delivery is a viscous solution.

14. The method of claim 7, wherein the composition formulated for enema delivery is administered once a day for at least 2 weeks.

15. The method of claim 9, wherein the composition formulated for enema delivery is administered in a dosage amount from about 0.1% w/w to 1.5% w/w per day for at least 2 weeks.

16. The method of claim 15, wherein the dosage amount is higher in the first week and reduced in the remaining weeks.

17. The method of claim 9, wherein the composition formulated for enema delivery further comprises additional components selected from the group consisting of preservative, viscosity enhancer/mucoadhesive, buffer, surfactant and solvent.

18. The method of claim 17, wherein the preservative is in an amount from about 0.1% w/w to about 1.5% w/w, the viscosity enhancer/mucoadhesive in an amount from about 0.5% w/w to about 5% w/w, and the buffer in an amount sufficient to adjust pH to between 6.5 and 7.5.

19. The method of claim 9, wherein metronidazole is the sole active agent in the composition formulated for enema delivery.

20. The method of claim 7, wherein said enema formulation consists essentially of metronidazole at a concentration of about 0.6% w/w in a pharmaceutically acceptable carrier.

21. The use of metronidazole in the amount from about 0.1% w/w to about 1.5% w/w in the manufacture of a medicament for the treatment of the symptoms of pouchitis or proctitis.
